(19) United States
(12) Patent Application Publication
(10) Pub. No.: US 2024/0138727 A1
(43) Pub. Date: May 2, 2024

(54) MUSCLE OXYGEN SATURATION DETECTION METHOD, AND MUSCLE OXYGEN SATURATION RECOVERY METHOD AND SYSTEM

(71) Applicant: SHENZHEN INSTITUTES OF ADVANCED TECHNOLOGY CHINESE ACADEMY OF SCIENCES, Shenzhen (CN)

(72) Inventors: Han CUI, Shenzhen (CN); Guanglin LI, Shenzhen (CN)

(73) Assignee: SHENZHEN INSTITUTES OF ADVANCED TECHNOLOGY CHINESE ACADEMY OF SCIENCES, Shenzhen (CN)

(21) Appl. No.: 18/399,430

(22) Filed: Dec. 28, 2023

Related U.S. Application Data
(63) Continuation of application No. PCT/CN2022/140076, filed on Dec. 19, 2022.

(30) Foreign Application Priority Data
Jan. 26, 2022 (CN) 202210096626.1

(51) Int. Cl. A61B 5/1455; A61B 5/00

(52) U.S. Cl. CPC A61B 5/14552; A61B 5/4519; A61B 5/4836; A61B 5/4848; A61B 5/7275; A61B 5/7282

(57) ABSTRACT

The present application is applicable to the technical field of muscle oxygen saturation detection, and provides a muscle oxygen saturation detection method, and a muscle oxygen saturation recovery method and system. The muscle oxygen saturation detection method is applied to a muscle oxygen saturation detection device. The present application combines pulse electrical stimulation for relieving the muscle fatigue and improving the muscle activity function with muscle oxygen saturation detection, and automatically triggers the turning-on and turning-off of electrical stimulation with the muscle oxygen saturation detection data of a wearer to form a closed-loop muscle function detection and stimulation system with feedback.

### CROSS-REFERENCE TO RELATED APPLICATIONS

The application claims priority to Chinese patent application No. 202210096626.1, filed on Jan. 26, 2022, the entire contents of which are incorporated herein by reference.

### TECHNICAL FIELD

The present application belongs to the technical field of muscle oxygen saturation detection, and particularly relates to a muscle oxygen saturation detection method, a muscle oxygen saturation recovery method and system, and a computer-readable storage medium.

### BACKGROUND

Muscle oxygen saturation (hereinafter referred to as SmO₂) represents the supply and consumption of local muscle blood flow.

A human body will consume oxygen in the muscle during exercise, which will lead to the continuous decline of the muscle oxygen saturation. That is, when the muscle oxygen saturation is low, it will be difficult for the muscle to maintain activities, and the human body must rest to increase the muscle oxygen saturation, otherwise the activity ability of the muscle will decrease constantly.

### SUMMARY

Embodiments of the present application provide a muscle oxygen saturation detection method, and a muscle oxygen saturation recovery method and system, which can solve the problem that it is difficult for the existing method to accurately calculate the muscle oxygen saturation.

In a first aspect, embodiments of the present application provide a muscle oxygen saturation detection method, applied to a muscle oxygen saturation detection device; the muscle oxygen saturation detection device includes a light source and at least two detectors, the light source is configured to alternately emit light of at least two different wavelengths, the at least two detectors include a first detector and a second detector, the distance between the first detector and the light source and the distance between the second detector and the light source are unequal; the muscle oxygen saturation detection method includes:
acquiring an intensity of light of a first wavelength exiting a tissue to be measured detected by the first detector to obtain a first exiting light intensity, and acquiring an intensity of the light of the first wavelength exiting the tissue to be measured detected by the second detector to obtain a second exiting light intensity when the light source emits light of the first wavelength to the tissue to be measured;
acquiring an intensity of light of a second wavelength exiting a tissue to be measured detected by the first detector to obtain a third exiting light intensity, and acquiring an intensity of the light of the second wavelength exiting the tissue to be measured detected by the second detector to obtain a fourth exiting light intensity when the light source emits light of the second wavelength to the tissue to be measured; and
determining a muscle oxygen saturation of the tissue to be measured according to an intensity of the light of the first wavelength incident on the tissue to be measured, an intensity of the light of the second wavelength incident on the tissue to be measured, the first exiting light intensity, the second exiting light intensity, the third exiting light, intensity, the fourth exiting light intensity, the distance between the first detector and the light source, and the distance between the second detector and the light source.

In a second aspect, embodiments of the present application provide a muscle oxygen saturation recovery method, applied to a recovery device, and including:
receiving information sent by a muscle oxygen saturation detection device, wherein the information includes a first notification for notifying the recovery device of turning-on or a second notification for notifying the recovery device of turning-off; and
performing a corresponding action based on the information, wherein a muscle oxygen saturation of a tissue to be measured detected by the muscle oxygen saturation detection device will rise when the recovery device is turned on.

In a third aspect, embodiments of the present application provide a muscle oxygen saturation recovery system, including a muscle oxygen saturation detection device and a recovery device; wherein
the muscle oxygen saturation detection device is configured to perform the muscle oxygen saturation detection method according to the first aspect;
the recovery device is configured to perform the muscle oxygen saturation recovery method according to the second aspect.

In a fourth aspect, embodiments of the present application provide an electronic device, including a memory, a processor, and a computer program stored in the memory and runnable on the processor, wherein the processor, when executing the computer program, implements the method according to the first aspect, or the processor, when executing the computer program, implements the method according to the second aspect.

In a fifth aspect, embodiments of the present application provide a computer-readable storage medium storing a computer program, wherein the computer program, when executed by a processor, implements the method according to the first aspect or the second aspect.

In a sixth aspect, embodiments of the present application provide a computer program product, wherein the computer program product, when running on an electronic device, causes the electronic device to perform the method according to the first aspect or the second aspect described above.

Compared with the prior art, the embodiments of the present application have the beneficial effects that:
since the distance between the first detector and the light source and the distance between the second detector and the light source are unequal, and the light intensities that the first detector and the second detector can detect are different when the distances from the light source are different. Therefore, after the light source alternately emits light of at least two different wavelengths, regardless of how much light is absorbed by the background (the background other than HbO₂ and Hb in the tissue to be measured), the muscle oxygen saturation of the tissue to be measured can be accurately calculated based on the light intensities detected by the first detector and the second detector, the intensity of the light incident on the tissue, the distance between the first detector and the light source, and the distance between the second detector and the light source. In this way, the user can judge whether to rest according to the calculated muscle oxygen saturation so as to recover the muscle oxygen saturation in time, thereby reducing the harm caused by the fact that the user continues to exercise without sufficient muscle oxygen saturation.

### BRIEF DESCRIPTION OF DRAWINGS

In order to more clearly illustrate the technical solutions in the embodiments of the present application, the accompanying drawings required to be used in the description of the embodiments or the prior art will be briefly introduced below.

FIG. 1 is a flowchart of a muscle oxygen saturation detection method according to an embodiment of the present application;

FIG. 2 is a schematic diagram of the positional relationship of one light source and two detectors according to an embodiment of the present application;

FIG. 3 is a flowchart of a muscle oxygen saturation recovery method according to an embodiment of the present application;

FIG. 4 is a waveform diagram of a symmetrical bidirectional pulse according to an embodiment of the present application;

FIG. 5 is a schematic circuit diagram of a recovery device according to another embodiment of the present application;

FIG. 6 is a structural block diagram of a muscle oxygen saturation recovery system according to an embodiment of the present application; and

FIG. 7 is a structural schematic diagram of an electronic device according to another embodiment of the present application.

### DETAILED DESCRIPTION OF THE EMBODIMENTS

In the following description, specific details, such as specific system structure and technology, are set forth for the purpose of explanation rather than limitation in order to provide a thorough understanding of embodiments of the present application. However, it will be apparent to one skilled in the art that the present application may be practiced in other embodiments without these specific details. In other instances, detailed descriptions of well-known systems, devices, circuits, and methods are omitted so as not to obscure the description of the present application with unnecessary details.

It should be understood that the term “including”, when used in the description and appended claims of the present application, indicates the presence of the described features, integers, steps, operations, elements and/or components, but does not exclude the presence or addition of one or more other features, integers, steps, operations, elements, components and/or groups thereof.

It should also be understood that the term “and/or” used in the description and appended claims of the present application refers to any combination and all possible combinations of one or more of the associated listed items, and includes these combinations.

In addition, in the description of the description and appended claims of the present application, the terms “first”, “second”, “third,” and the like are used merely to distinguish descriptions and are not to be construed as indicating or implying relative importance.

Reference to “one embodiment” or “some embodiments” and the like described in description of the present application means that a specific feature, structure or characteristic described in connection with this embodiment is included in one or more embodiments of the present application. Thus, the phrases “in one embodiment”, “in some embodiments”, “in other embodiments” and “in further embodiments” appearing in different places in this description do not necessarily all refer to the same embodiment, but mean “one or more but not all embodiments” unless otherwise emphasized.

In an embodiment of the present application, muscle oxygen saturation detection is performed on a tissue to be measured using a muscle oxygen saturation detection device including a light source and at least two detectors. The light source is configured to alternately emit light of at least two different wavelengths, the at least two detectors include a first detector and a second detector, and the distance between the first detector and the light source and the distance between the second detector and the light source are unequal. Every time when the light source emits light to the tissue to be measured, the first detector and the second detector each detect a corresponding light intensity from the tissue to be measured. When the first detector and the second detector both acquire the light intensities corresponding to the two different wavelengths, the muscle oxygen saturation of the tissue to be measured is calculated based on the light intensities acquired by the two detectors, the light intensities of the light of the two different wavelengths incident on the tissue, and the distances between the two detectors and the light source. By the above calculation method, the muscle oxygen saturation of the tissue to be measured can be accurately calculated regardless of how much light is absorbed by the background (the background except HbO₂ and Hb in the tissue to be measured).

### Embodiment 1

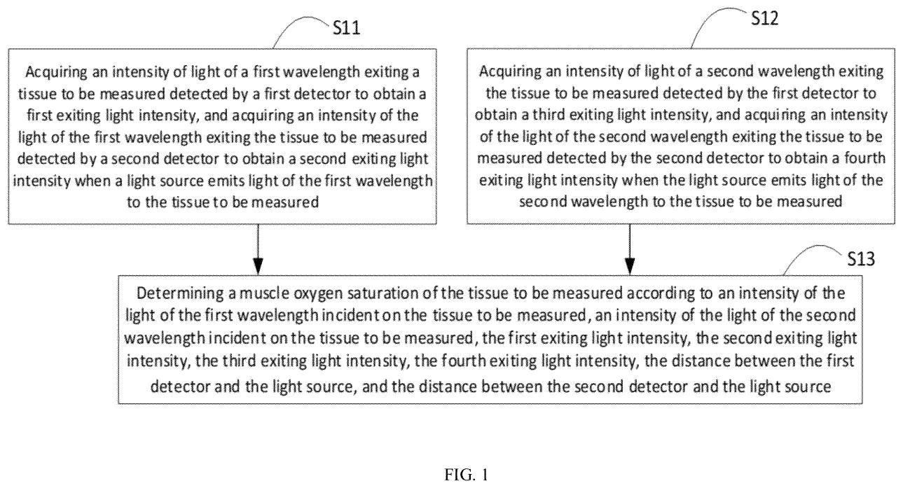

FIG. 1 shows a flowchart of a muscle oxygen saturation detection method according to an embodiment of the present application, the method is applied to a muscle oxygen saturation detection device, and the muscle oxygen saturation detection device may be a wearable device.

The muscle oxygen saturation detection device of an embodiment of the present application includes a light source and at least two detectors. The light source is configured to alternately emit light of at least two different wavelengths, and the distance between the first detector and the light source and the distance between the second detector and the light source are unequal. Description will be in detail made below by taking a muscle oxygen saturation detection device including two detectors (a first detector and a second detector) and one light source as an example:

Step S11, acquiring an intensity of light of a first wavelength exiting a tissue to be measured detected by the first detector to obtain a first exiting light intensity, and acquiring an intensity of the light of the first wavelength exiting the tissue to be measured detected by the second detector to obtain a second exiting light intensity when the light source emits light of the first wavelength to the tissue to be measured.

The tissue to be measured is a tissue of which the muscle oxygen saturation needs to be measured.

Both the first detector and the second detector described above may be a photodetector (PD).

In an embodiment of the present application, the light source is configured to alternately emit light of at least two different wavelengths.

In some embodiments, light of different wavelengths is emitted by different light emitting diodes (LEDs) for ease of control. For example, when the light source includes two LEDs, near-infrared light of two different wavelengths is alternately emitted by the two LEDs.

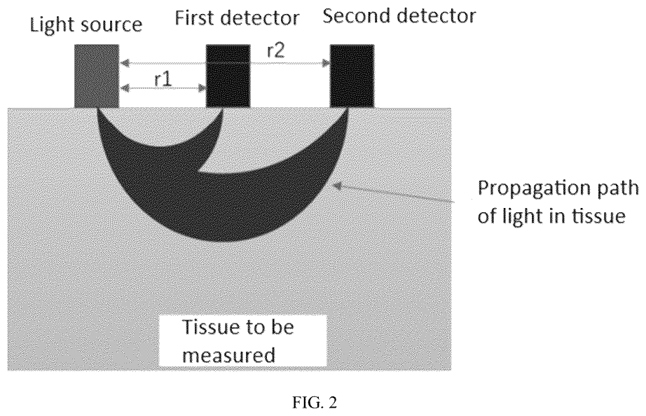

FIG. 2 shows a schematic diagram of the positional relationship of one light source and two detectors according to an embodiment of the present application. In this embodiment, after the light source emits light, the light scatters and propagates in the tissue to be measured to form an arc-shaped propagation path, which is detected by the PDs far away from the light source, and the farther the PD away from the light source, the deeper the detected light intensity signal, as shown in FIG. 2.

Step S12, acquiring an intensity of light of a second wavelength exiting the tissue to be measured detected by the first detector to obtain a third exiting light intensity, and acquiring an intensity of the light of the second wavelength exiting the tissue to be measured detected by the second detector to obtain a fourth exiting light intensity when the light source emits light of the second wavelength to the tissue to be measured.

Step S13, determining a muscle oxygen saturation of the tissue to be measured according to an intensity of the light of the first wavelength incident on the tissue to be measured, an intensity of the light of the second wavelength incident on the tissue to be measured, the first exiting light intensity, the second exiting light intensity, the third exiting light intensity, the fourth exiting light intensity, the distance between the first detector and the light source, and the distance between the second detector and the light source.

According to the analysis of the inventor, the detected light intensity (or light intensity signal) comes from the scattering and absorption of the incident light intensity signal in the tissue to be measured, and the scattering and absorption of light of different wavelengths in the tissue to be measured are different. Oxygenated hemoglobin (HbO₂) and deoxygenated hemoglobin (Hb) are the main light absorption chromophores of the tissue to be measured, and the relationship between the light intensity signals detected based on the irradiation of light of the two wavelengths and the characteristic parameters of the tissue to be measured is as fellows:

$$\frac{{I}_{λ 1}^{′}}{{I}_{λ 1}} = ( {ε}_{{HbO}_{2}}^{λ 1} {C}_{{HbO}_{2}} + {ε}_{Hb}^{λ 1} {C}_{Hb} ) \cdot r \cdot DPF + {G}^{λ 1} \\ \frac{{I}_{λ 2}^{′}}{{I}_{λ 2}} = ( {ε}_{{HbO}_{2}}^{λ 2} {C}_{{HbO}_{2}} + {ε}_{Hb}^{λ 2} {C}_{Hb} ) \cdot r \cdot DPF + {G}^{λ 2}$$

Where I′ and I represent the light intensities of the exiting light and the incident light, respectively, ε_{HbO}_{₂} and ε_{Hb} represent the light absorption coefficients of HbO₂ and Hb, respectively, C_{HbO}_{₂} and C_{Hb} are the concentrations of HbO₂ and Hb, r is the distance between the light source and the detector, DPF is the weight coefficient of the distance r, also called differential path factor, and G is the background absorption in the tissue to be measured except for HbO₂ and Hb. In the above equation, “●” denotes the sign of the multiplication operation, the superscripts and subscripts λ1 and λ2 denote light of two different wavelengths, respectively, C_{HbO}_{₂} and C_{Hb} are the parameters to be solved, and other parameters are known except G. Thus, when light of two wavelengths is used to alternately illuminate the tissue to be measured, since the absorption of the light of two wavelengths in the tissue to be measured is different, two equations can be obtained to solve the unknowns C_{HbO}_{₂} and C_{Hb}. However, due to the presence of the unknown G, G can only be eliminated from the set of variances by calculating the temporal variation ΔC_{HbO}_{₂} of C_{HbO}_{₂} and the temporal variation ΔC_{Hb} of C_{Hb}, but the specific value corresponding to the oxygen saturation cannot be obtained from ΔC_{HbO}_{₂} and ΔC_{Hb}. In order to obtain a specific value corresponding to the oxygen saturation, an embodiment of the present application uses a method adopting two detectors (a first detector and a second detector) to calculate the oxygen saturation. As shown in FIG. 2, the light intensity signals obtained by the detectors at two positions distanced from the light sources by r1 and r2 are subtracted. Because the background absorption of the two detection distances is not much different at the same wavelength, it can be assumed that G is equal, and then the following equation is obtained:

$$Δ \frac{{I}_{λ 1}^{′}}{{I}_{λ 1}} = ( {ε}_{{HbO}_{2}}^{λ 1} {C}_{{HbO}_{2}} + {ε}_{Hb}^{λ 1} {C}_{Hb} ) \cdot ( r 2 - r 1 ) \cdot DPF \\ Δ \frac{{I}_{λ 2}^{′}}{{I}_{λ 2}} = ( {ε}_{{HbO}_{2}}^{λ 2} {C}_{{HbO}_{2}} + {ε}_{Hb}^{λ 2} {C}_{Hb} ) \cdot ( r 2 - r 1 ) \cdot DPF$$

Where r2 and r1 are the distances of the second detector and the first detector from the light source, respectively.

$$Δ \frac{{I}_{λ 1}^{′}}{{I}_{λ 1}} and Δ \frac{{I}_{λ 2}^{′}}{{I}_{λ 2}}$$

are the ratio of the exiting light intensity to the incident light intensity detected by the second detector minus the ratio of the exiting light intensity to the incident light intensity detected by the first detector under the irradiation condition of λ1 light and λ2 light, respectively. There is no longer an unknown term G in this equation, so values corresponding to the concentrations C_{HbO}_{₂} and C_{Hb} of HbO₂ and Hb can be obtained.

In some embodiments, the muscle oxygen saturation may be calculated in the following manner:

$${rSO}_{2} = \frac{{C}_{{HbO}_{2}}}{{C}_{{HbO}_{2}} + {C}_{Hb}}$$

In an embodiment of the present application, the distance between the first detector and the light source and the distance between the second detector and the light source are unequal, and when the first detector and the second detector are at different distances from the light source, the light intensities that can be detected by the first detector and the second detector are also different, thus, after the light source alternately emits light of at least two different wavelengths, no matter how much light is absorbed by the background (the background other than HbO₂ and Hb in the tissue to be measured), the concentration of oxygenated hemoglobin and the concentration of deoxygenated hemoglobin of the tissue to be measured can be accurately calculated according to the light intensities detected by the first detector and the second detector, the intensity of light emitted by the light source, and the distance of the first detector and the second detector from the light source, thereby improving the accuracy of the calculated muscle oxygen saturation of the tissue to be measured according to the concentration of oxygenated hemoglobin and the concentration of deoxygenated hemoglobin. Thus, the user can judge, according to the calculated muscle oxygen saturation, whether to rest so as to recover the muscle oxygen saturation, thereby reducing the harm caused by the fact that the user continues to exercise without sufficient muscle oxygen saturation.

In some embodiments, in order to increase the muscle oxygen saturation of the tissue to be measured as soon as possible, after the above step S14, the method further includes:
judging whether a turning-on condition of the recovery device is satisfied based on the muscle oxygen saturation and a first preset threshold, and notifying the recovery device of turning-on when the turning-on condition of the recovery device is satisfied, wherein the muscle oxygen saturation of the tissue to be measured will rise when the recovery device is turned on.

In some embodiments, the first preset threshold may be a value related to an average value of rSO₂ (assumed to be r̅S̅O̅₂̅) of the tissue to be measured in a certain time range when the human body is in a non-exercise state, and the first preset threshold is less than r̅S̅O̅₂̅, wherein,

$$\overline{{rSO}_{2}} = \frac{\sum {rSO}_{2} ( {t}_{i} )}{\sum {t}_{i}}$$

The above-mentioned r̅S̅O̅₂̅ is also referred to as a baseline value (i.e., the average value of rSO₂ in a certain time range), and tᵢ represents the time point during the baseline acquisition.

At this time, the detected muscle oxygen saturation may be compared with a certain proportion of r̅S̅O̅₂̅ (assumed to be

$$\frac{\overline{{rSO}_{2}}}{2}$$

which is the first preset threshold), and if the detected muscle oxygen saturation is less than

$$\frac{\overline{{rSO}_{2}}}{2} ,$$

it is determined that the turning-on condition of the recovery device is currently satisfied.

In some embodiments, the first preset threshold may be a preset value, and is assumed to be α, the derivative of the slope of the detected muscle oxygen saturation is calculated, the derivative is compared with α, and if the derivative is less than α, as shown in the following equation

$$\frac{{rSO}_{2}^{′} ( {t}_{i} ) - {rSO}_{2}^{′} ( {t}_{i - 1} )}{{t}_{i} - {t}_{i - 1}} < α ,$$

it is determined that the turning-on condition of the recovery device is currently met. Where rSO′₂(tᵢ) represents the slope of rSO₂ and

$$\frac{{rSO}_{2}^{′} ( {t}_{i} ) - {rSO}_{2}^{′} ( {t}_{i - 1} )}{{t}_{i} - {t}_{i - 1}}$$

represents the derivative of the slope of rSO₂. In some embodiments, α may be set to a value less than 0 considering that the derivative of the slope of rSO₂ is less than 0 when the recovery device needs to be turned on.

In an embodiment of the present application, the muscle oxygen saturation detection device and the recovery device may form a closed-loop muscle oxygen saturation detection and stimulation system with feedback. Specifically, the muscle oxygen saturation detection device may send a notification including information of turning on the recovery device to the recovery device when determining that the turning-on condition of the recovery device is satisfied and the recovery device is automatically turned on after receiving the notification to perform a corresponding operation on the tissue to be measured, thereby increasing the muscle oxygen saturation of the tissue to be measured.

In some embodiments, in order to save the resources of the recovery device, after notifying the recovery device of turning-on, the method further includes:
continuing to determine a muscle oxygen saturation of the tissue to be measured, judging whether a turning-off condition of the recovery device is satisfied based on the continuously determined muscle oxygen saturation and a second preset threshold, and notifying the recovery device of turning-off when the turning-off condition of the recovery device is satisfied.

In some embodiments, when the first preset threshold is a value related to r̅S̅O̅₂̅, the second preset threshold is also a value related to r̅S̅O̅₂̅, with the difference that the second preset threshold is greater than the first preset threshold, and the second preset threshold is less than or equal to r̅S̅O̅₂̅. For example, if the first preset threshold is

$$\frac{\overline{{rSO}_{2}}}{2} ,$$

the second preset threshold may be 0.9r̅S̅O̅₂̅. At this time, when the muscle oxygen saturation detection device determines that the detected muscle oxygen saturation is greater than the second preset threshold, it is determined that the turning-off condition of the recovery device is satisfied, i.e., the recovery device is notified of turning-off when the following formula holds: rSO₂(tᵢ)>0.9r̅S̅O̅₂̅.

In other embodiments, when the first preset threshold is a value unrelated to r̅S̅O̅₂̅, the second preset threshold is also a value unrelated to r̅S̅O̅₂̅, with the difference that the first preset threshold is less than the second preset threshold (the second preset threshold may be set to a value greater than zero). Assuming that the second preset threshold is , it is determined that the turning-off condition for the recovery device is satisfied when the following formula is satisfied:

$$\frac{{rSO}_{2}^{′} ( {t}_{i} ) - {rSO}_{2}^{′} ( {t}_{i - 1} )}{{t}_{i} - {t}_{i - 1}} < β$$

In an embodiment of the present application, the muscle oxygen saturation of the tissue to be measured will rise when the recovery device is turned on. When the muscle oxygen saturation detection device detects that the muscle oxygen saturation in the tissue to be measured is sufficient, the recovery device is notified of turning-off, thereby saving the resources of the recovery device.

In the above embodiment, the muscle oxygen saturation detection device itself judges whether the turning-on condition of the recovery device is currently satisfied, or judges whether the turning-off condition of the recovery device is currently satisfied, and notifies the recovery device of turning-on or turning-off according to the judgment result, and in some embodiments, the muscle oxygen saturation detection device directly sends the detected muscle oxygen saturation to the recovery device, and the recovery device itself judges whether to be turned on or off.

In some embodiments, in order to rapidly increase the muscle oxygen saturation of the tissue to be measured, after notifying the recovery device of turning-on, the method further includes:
continuing to determine a muscle oxygen saturation of the tissue to be measured, judging whether an early warning condition is met based on the muscle oxygen saturation within the preset time period continuously determined and the preset rising amplitude, and issuing an early warning prompt when the early warning condition is satisfied, wherein the early warning prompt includes information for indicating that the rising amplitude of the muscle oxygen saturation is too slow.

In the embodiment of the present application, after the recovery device is turned on, the muscle oxygen saturation detection device continues to perform muscle oxygen saturation detection on the tissue to be measured, compares the rising amplitude of the muscle oxygen saturation detected within the preset time period with a preset rising amplitude, and if the rising amplitude is less than the preset rising amplitude, determines that the early warning condition is satisfied, and sends an early warning prompt. The early warning prompt may be provided by text or by sound/light, such as a buzzer.

In some embodiments. the preset time period is smaller, such as 1 minute, that is, it is judged whether the rising amplitude of the muscle oxygen saturation within one minute after the recovery device is turned on satisfies the early warning condition. Since the preset time period is smaller, it can be judged more quickly whether the early warning condition is currently satisfied, so that the user can know that the rising amplitude of the muscle oxygen saturation does not meet the requirement more in time.

In some embodiments, the early warning prompt described above may also include information for instructing the user to increase stimulation, parameters or stop exercising. After the stimulation parameters are increased, the rising amplitude of the muscle oxygen saturation of the tissue to be measured is increased. Since the early warning prompt includes information for instructing the user to increase the stimulation parameters or stop exercising, the user can choose to increase the stimulation parameters of the recovery device or to stop exercising according to the early warning prompt, thereby increasing the muscle oxygen saturation of the tissue to be measured in time.

In some embodiments, after the muscle oxygen saturation detection device determines that the early warning condition is met, the recovery device is notified to increase the stimulation parameters. Since the muscle oxygen saturation detection device can automatically notify the recovery device of increasing of the stimulation parameters, the user operation is reduced, and the good experience of the user is improved.

To sum up, the muscle oxygen saturation detection method provided by the embodiment of the present application can accurately calculate the concentration of oxygenated hemoglobin and the concentration of deoxygenated hemoglobin of the tissue to be measured, thereby improving the accuracy of the muscle oxygen saturation of the tissue to be measured calculated according to the concentration of oxygenated hemoglobin and the concentration of deoxygenated hemoglobin.

Further, since the muscle oxygen saturation detection device is also in communication with the recovery device, the muscle oxygen saturation detection method provided by the embodiment of the present application can also notify the recovery device of turning-on or turning-off, so that the muscle oxygen saturation of the tissue to be measured can be guaranteed to meet the needs of the user.

Further, since the muscle oxygen saturation detection device further includes a buzzer, the muscle oxygen saturation detection method provided by the embodiment of the present application can also issue an early warning prompt including information for indicating that the rising amplitude of the muscle oxygen saturation is too slow.

It should be understood that the magnitudes of the sequence numbers of the various steps in the above-described embodiment do not imply the order of execution, and the order of execution of the various processes should be determined by their functions and intrinsic logic, and should not constitute any limitation to the implementation of the embodiment of the present application.

### Embodiment 2

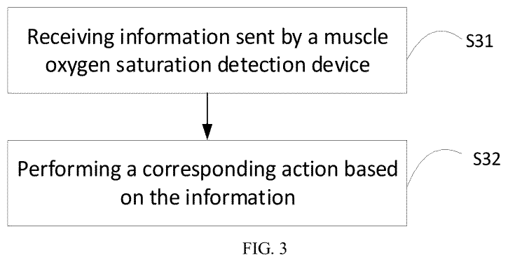

FIG. 3 shows a flowchart of a muscle oxygen saturation recovery method provided by an embodiment of the present application, and the muscle oxygen saturation recovery method is applied to a recovery device and will be described in detail below:

Step S31, receiving information sent by a muscle oxygen saturation detection device, wherein the information includes a first notification for notifying the recovery device of turning-on or a second notification for notifying the recovery device of turning-off.

In embodiments of the present application, the muscle oxygen saturation detection device may communicate via wireless communication.

Specifically, when the muscle oxygen saturation detection device determines that the muscle oxygen saturation of the tissue to be measured needs to be increased by an external device, information including a first notification will be sent to the recovery device; conversely, when the muscle oxygen saturation detection device determines that the muscle oxygen saturation of the tissue to be measured does not need to be increased by an external device, information including a second notification is sent to the recovery device.

Step S32, performing a corresponding action based on the information, wherein when the recovery device is turned on, the muscle oxygen saturation of the tissue to be measured detected by the muscle oxygen saturation detection device will rise.

Specifically, if the recovery device receives the information including the first notification, a turning-on action is performed; or, if the recovery device receives the information including the second notification, a turning-off action is performed.

In the embodiment of the present application, the recovery device can perform a corresponding action based on the information sent by the muscle oxygen saturation detection device, and the muscle oxygen saturation of the tissue to be measured will rise when the recovery device is turned on, so that the muscle oxygen saturation of the tissue to be measured can be increased by the recovery device, thereby reducing the risk of the human body occurring due to lower muscle oxygen saturation.

In some embodiments, the recovery device includes a device having at least one of the following functions: electrical stimulation, mechanical massage, heating, ultrasound, electromagnetism, illumination, and the like.

In the embodiment of the present application, when the tissue is treated by electrical stimulation, mechanical massage, heat, ultrasound, electromagnetism, and/or illumination, the muscle oxygen saturation of the tissue to be measured will rise.

In some embodiments, if the recovery device has the function of electrical stimulation, the above step S32 includes:
performing a turning-on action and outputting an electrical pulse to the tissue to be measured if the information includes a first notification.

In the embodiment of the present application, the recovery device electrically stimulates the tissue to be measured by outputting the electrical pulse to the tissue to increase the muscle oxygen saturation of the tissue to be measured.

In some embodiments, performing the turning-on action and outputting the electrical pulse to the tissue to be measured includes:

A1, performing a turning-on action, and acquiring a target amplitude, a target period and a target pulse width.

A2, outputting a symmetrical bidirectional electrical pulse to the tissue to be measured according to the target amplitude, the target period and the target pulse width.

Specifically, the amplitude, period and pulse width of the symmetrical bidirectional electrical pulse are equal to the target amplitude, the target period and the target pulse width, respectively.

In the embodiment of the present application, the recovery device outputs the symmetrical bidirectional pulse, i.e. alternately generates electrical pulses of equal amplitude and opposite direction, wherein the negative-going pulse is primarily used to generate stimulation, and the positive-going pulse is primarily used to recover the charge at the stimulation site. Since the symmetrical bidirectional pulse can reduce the charge accumulation at the stimulation site, the tissue to be measured is protected from damage.

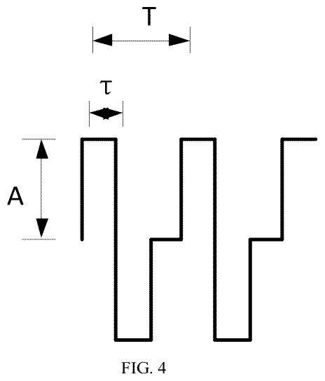

The waveform diagram of the symmetrical bidirectional pulse is shown in FIG. 4, where A represents the amplitude, T represents the period of stimulation, and τ represents the pulse width, these three parameters can be adjusted according to the instruction of the user, or the information fed back by the muscle oxygen saturation detection device to change the magnitude of the stimulation amount. For example, the information fed back by the muscle oxygen saturation detection device may include values of A, T and τ, so that the recovery device can be adjusted based on the values included in the information.

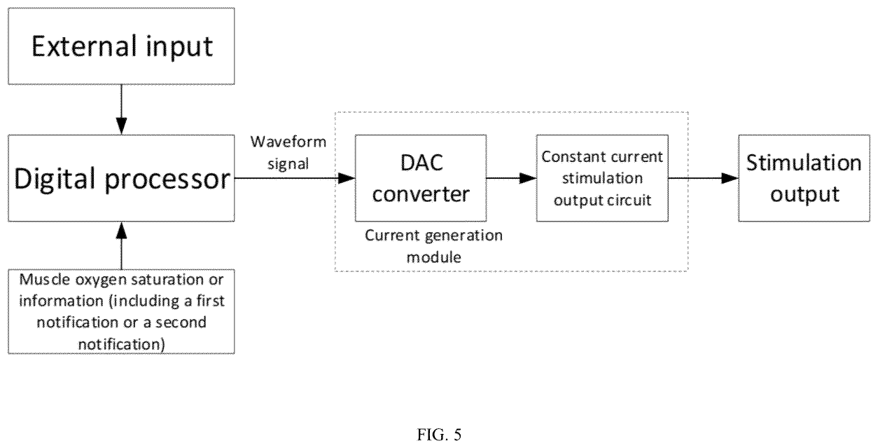

The circuit of the recovery device includes a digital processor and a current generation module, as shown in FIG. 5. The digital processor receives the muscle oxygen saturation arid controls the turning-on or turning-off of the current generation module based on the muscle oxygen saturation, or the digital processor receives information including the first notification or the second notification and controls the turning-on or turning-off of the current generation module based on the information. The digital processor may also interact with the outside, such as a user, to receive externally input stimulation parameters or turning-on or turning-off information. The digital processor is responsible for inputting the received stimulation parameters into the current generation module, causing the current generation module to generate a waveform according to the stimulation parameters. The current generation module of this embodiment includes a digital to analog converter (DAC) and a constant current stimulation output circuit. The DAC converter converts pulse information input by the digital processor into a signal represented by an analog quantity. The constant current stimulation output circuit is used to ensure that the stimulation current remains at a stable level as the resistance changes. Since the resistance of human tissue skin varies from location to location, it is advantageous for the current generation module to include the constant current stimulation output circuit to ensure the stability of the output current, thereby improving the good experience of the user.

It should be understood that the magnitudes of the sequence numbers of the various steps in the above-described embodiment do not imply the order of execution, and the order of execution of the various processes should be determined by their functions and intrinsic logic, and should not constitute any limitation to the implementation of the embodiment of the present application.

### Embodiment 3

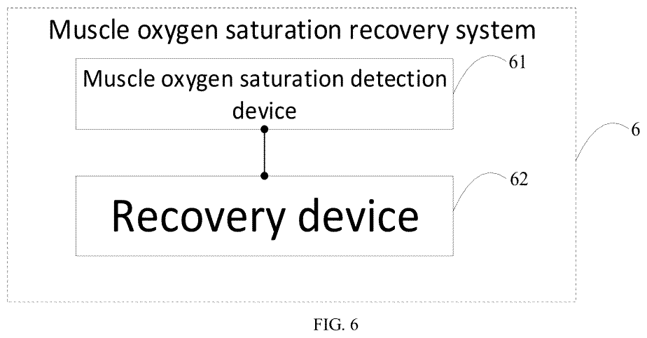

Corresponding to the method in the above embodiment, FIG. 6 shows a structural block diagram of a muscle oxygen saturation recovery system according to an embodiment of the present application, and only the parts relevant to the embodiment of the present application are shown for ease of explanation.

With reference to FIG. 6, the muscle oxygen saturation recovery system 6 includes a muscle oxygen saturation detection device 61 and a recovery device 62.

The muscle oxygen saturation detection device 61 is configured to perform the steps of the muscle oxygen saturation detection method in Embodiment 1, and will not be described in detail here.

The recovery device 62 is configured to perform the steps of the muscle oxygen saturation recovery method in Embodiment 2, and will not be described in detail here.

It should be noted that the information interaction, execution process, and other content between the above devices are based on the same idea as the method embodiments of the present application, and the specific functions and technical effects thereof can refer to the method embodiments, and will not be described in detail here.

### Embodiment 4

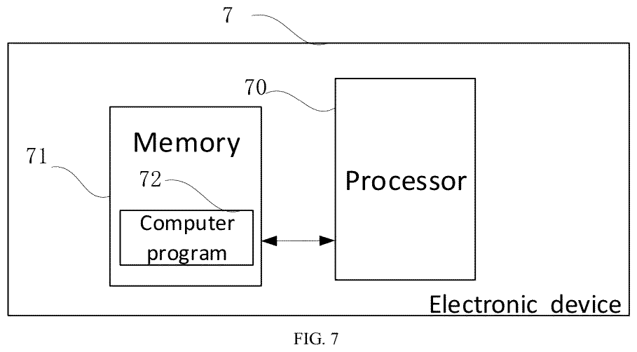

FIG. 7 is a structural schematic diagram of an electronic device according to an embodiment of the present application. As shown in FIG. 7, the electronic device 7 of this embodiment includes at least one processor 70 (only one processor is shown in FIG. 7), a memory 71, and a computer program 72 stored in the memory 71 and runnable on the at least one processor 70. The processor 70, when executing the computer program 72, is configured to implement the steps of the various method embodiments described above.

In some embodiments, the electronic device 7 further includes a light source and at least two detectors when the computer program 72 executes the processor 70 to implement the steps in the method embodiment 1.

It will be appreciated by those skilled in the art that FIG. 7 is merely an example of the electronic device 7 and does not constitute a limitation to the electronic device 7, and may include more or fewer components than illustrated, or may combine some components, or different components, such as input/output devices and network access devices.

The processor 70 may be a central processing unit (CPU), and may also be another general-purpose processor, a digital signal processor (DSP), an application specific integrated circuit (ASIC), a field-programmable gate array (FPGA) or other programmable logic devices, discrete gates or transistor logic devices, discrete hardware components, or the like. The general-purpose processor may be a microprocessor or any conventional processor or the like.

The memory 71 may in some embodiments be an internal storage unit of the electronic device 7, for example a hard disk or a memory of the electronic device 7. The memory 71 may in other embodiments also be an external storage device of the electronic device 7, such as a plug-in hard disk, a smart media card (SMC), a secure digital (SD) card and a flash card provided on the electronic device 7. Further, the memory 71 may also include both an internal storage unit and an external storage device of the electronic device 7. The memory 71 is configured to store the operating system, application programs, BootLoader, data and other programs, such as the program code of the computer program. The memory 71 may also be used to temporarily store data that has been output or is to be output.

Those skilled in the art can clearly appreciate that for convenience and brevity of description, only the division of the above-described functional units or modules is illustrated, in practice, the above-described functions may be distributed by different functional units or modules, i.e., the internal structure of the device may be divided into different functional units or modules as needed to perform all or part of the above-described functions. The functional units or modules in the embodiments may be integrated in one processing unit, or the units may be physically present separately, or two or more units may be integrated in one unit, and the integrated unit may be implemented in the form of hardware or software functional units. In addition, the specific names of the respective functional units and modules are also only for convenience of distinguishing from each other, and are not used to limit the protection scope of the present application. The specific working processes of the units and modules in the above system can refer to the corresponding processes in the foregoing method embodiments, and will not be repeated here.

An embodiment of the present application also provides a network device including at least one processor, a memory, and a computer program stored in the memory and runnable on the at least one processor, wherein the processor, when executing the computer program, implements the steps in any of the various method embodiments described above.

An embodiment of the present application further provides a computer-readable storage medium, which stores a computer program, and the computer program, when executed by a processor, implements the steps in the above-described method embodiments.

An embodiment of the present application provides a computer program product, wherein the computer program product, when running on an electronic device, causes the electronic device to implement the steps in the method embodiments described above.

The integrated unit, if implemented in the form of a software functional unit and sold or used as a stand-alone product, may be stored in a computer-readable storage medium. Based on such understanding, all or part of the processes of the present application implementing the method embodiments may be performed by instructing associated hardware through a computer program which may be stored on a computer-readable storage medium, and the computer program, when executed by a processor, can implement the steps of the above-described method embodiments. The computer program includes a computer program code, which may be a source code, an object code, an executable file or some intermediate files, etc. The computer readable medium may include at least any entity or device capable of carrying the computer program code to photographing devices/electronic devices, a recording medium, a computer memory, a read-only memory (ROM), a random access memory (RAM), an electrical carrier wave signal, a telecommunication signal, and a software distribution medium, such as a USB flash disk, a removable hard disk, a magnetic disk or an optical disk. In some jurisdictions, according to legislation and patent practice, the computer-readable medium cannot be an electrical carrier wave signal or a telecommunication signal.

In the above-described embodiments, the description of each embodiment has its own emphasis. For the parts that are not detailed or recorded in one embodiment, please refer to the relevant descriptions of other embodiments.

A person having ordinary skill in the art will appreciate that the units and algorithm steps of the various embodiments described in connection with the embodiments disclosed here may be implemented in electronic hardware, or a combination of computer software and electronic hardware. Whether these functions are implemented as hardware or software depends upon the specific application and design constraints of the technical solution. Skilled professionals may use different methods to implement the described functions for each specific application, but such implementation should not be considered as beyond the scope of the present application.

In the embodiments provided by the present application, it should be understood that the disclosed device/network device and method may be implemented in other ways. For example, the device/network device embodiments described above are merely illustrative, for example, the division of modules or units is only a logical function division, and other division methods may be implemented in practice, for example, multiple units or components may be combined or integrated into another system, or some features may be omitted or not implemented. Further, the coupling or direct coupling or communication connections shown or discussed may be indirect coupling or communication connections through some interfaces, devices or units, which may be electrical, mechanical or other forms.

The units illustrated as separate components may or may not be physically separated, and the components shown as units may or may not be physical units, i.e. may be located at one place, or may be distributed over a plurality of network units. Some or all of the units may be selected according to actual needs to achieve the object of the embodiment.

The embodiments described above are only used to illustrate but not to limit the technical solutions of the present application; although the present application has been described in detail with reference to the foregoing embodiments, it will be understood by a person having ordinary skill in the art that the technical solutions described in the foregoing embodiments can still be modified or some technical features in the technical solutions are replaced equivalently; however, such modifications or replacements, which do not make the essence of the corresponding technical solutions depart from the spirit and scope of the technical solutions of the embodiments of the present application, shall be included in the protection scope of the present application.

What is claimed is:

1. A muscle oxygen saturation detection method, applied to a muscle oxygen saturation detection device, wherein the muscle oxygen saturation detection device comprises a light source and at least two detectors, the light source is configured to alternately emit light of at least two different wavelengths, the at least two detectors comprise a first detector and a second detector, a distance between the first detector and the light source and a distance between the second detector and the light source are unequal, and the muscle oxygen saturation detection method comprises:
acquiring an intensity of light of a first wavelength exiting a tissue to be measured detected by the first defector to obtain a first exiting light intensity, and acquiring an intensity of the light of the first wavelength exiting the tissue to be measured detected by the second detector to obtain a second exiting light intensity when the light source emits light of the first wavelength to the tissue to be measured;
acquiring an intensity of light of a second wavelength exiting a tissue to be measured detected by the first detector to obtain a third exiting light intensity, and acquiring an intensity of the light of the second wavelength exiting the tissue to be measured detected by the second detector to obtain a fourth exiting light intensity when the light source emits light of the second wavelength to the tissue to be measured; and
determining a muscle oxygen saturation of the tissue to be measured according to an intensity of the light of the first wavelength incident on the tissue to be measured, an intensity of the light of the second wavelength incident on the tissue to be measured, the first exiting light intensity, the second exiting light intensity, the third exiting light intensity, the fourth exiting light intensity, the distance between the first detector and the light source, and the distance between the second detector and the light source.

2. The muscle oxygen saturation detection method according to claim 1, wherein after determining the muscle oxygen saturation of the tissue to be measured, the method further comprises:
judging whether a turning-on condition of a recovery device is satisfied based on the muscle oxygen saturation and a first preset threshold, and notifying the recovery device of turning-on when the turning-on condition of the recovery device is satisfied, wherein the muscle oxygen saturation of the tissue to be measured will rise when the recovery device is turned on.

3. The muscle oxygen saturation detection method according to claim 2, wherein after notifying the recovery device of turning-on, the method further comprises:
continuing to determine a muscle oxygen saturation of the tissue to be measured, judging whether a turning-off condition of the recovery device is satisfied based on the continuously determined muscle oxygen saturation and a second preset threshold, and notifying the recovery device of turning-off when the turning-off condition of the recover device is satisfied.

4. The muscle oxygen saturation detection method according to claim 2, wherein after notifying the recovery device of turning-on, the method further comprises:
continuing to determine a muscle oxygen saturation of the tissue to be measured, judging whether an early warning condition is satisfied based on the continuously determined muscle oxygen saturation within a preset time period, and a preset rising amplitude, and issuing an early warning prompt when the early warning condition is satisfied, wherein the early warning prompt comprises information for indicating that a rising amplitude of the muscle oxygen saturation is too slow.

5. A muscle oxygen saturation recovery method, applied to a recovery device, and comprising:
receiving information sent by a muscle oxygen saturation detection device, wherein the information comprises a first notification for notifying the recovery device of turning-on or a second notification for notifying the recovery device of turning-off; and
performing a corresponding action based on the information, wherein a muscle oxygen saturation of a tissue to be measured detected by the muscle oxygen saturation detection device will rise when the recovery device is turned on.

6. The muscle oxygen saturation recovery method according to claim 5, wherein performing the corresponding action based on the information comprises:
performing a turning-on action and outputting an electrical pulse to the tissue to be measured under the condition that the information comprises the first notification.

7. The muscle oxygen saturation recovery method according to claim 6, wherein performing the turning-on action and outputting the electrical pulse to the tissue to be measured comprises:
performing a turning-on action and acquiring a target amplitude, a target period and a target pulse width; and
outputting a symmetrical bidirectional electrical pulse to the tissue to be measured according to the target amplitude, the target period and the target pulse width.

8. A muscle oxygen saturation recovery system, comprising a muscle oxygen saturation detection device and a recovery device; wherein
the muscle oxygen saturation detection device is configured to perform the muscle oxygen saturation detection method according to claim 1;
the recovery device is configured to perform the muscle oxygen saturation recovery method according to claim 5.
